(19) United States
(12) Patent Application Publication
(10) Pub. No.: US 2026/0369398 A1
(43) Pub. Date: Nov. 19, 2026

(54) SYSTEM AND METHOD FOR SURGICAL TRAY TRACKING

(71) Applicant: TRAY IQ LLC, Columbia, MO (US)

(72) Inventor: Frank T. Baumstark, JR., Columbia, MO (US)

(21) Appl. No.: 19/753,228

(22) Filed: Jul. 24, 2026

Related U.S. Application Data
(63) Continuation-in-part of application No. 19/539,497, filed on Feb. 13, 2026.
(60) Provisional application No. 63/801,244, filed on May 7, 2025.

(51) Int. Cl. G16H 40/40; A61B 50/33

(52) U.S. Cl. CPC G16H 40/40; A61B 50/33

(57) ABSTRACT

A system and computer-implemented method for tracking a surgical tray within a healthcare facility, including receiving from a sensor tag coupled to the surgical tray location data and a time series of temperature data, determining a workflow state of the surgical tray based on the location data, and computing a processing-exposure value based on the time series of temperature data. The computer-implemented method further includes generating cryptographic proof indicating that the processing-exposure value satisfies a predetermined threshold, without revealing the time series of temperature data or the processing-exposure value. The cryptographic proof and a chain-of-custody record associated with the surgical tray are recorded on a distributed ledger, which recording may trigger a pre-determined action such as a lifecycle or workflow transition, a financial transaction, an operational reaction, and/or a notification.

### CROSS-REFERENCE TO RELATED APPLICATIONS

The current patent application is a continuation-in-part non-provisional utility patent application which claims priority benefit, with regard to all common subject matter, of earlier-filed U.S. patent application Ser. No. 19/539,497; titled “SURGICAL TRAY TRACKER”; and filed Feb. 13, 2026. The earlier-filed patent application is hereby incorporated by reference, in its entirety, into the current patent application.

The parent patent application noted above (19/539,497) claims priority benefit, with regard to all common subject matter, of earlier-filed U.S. Provisional Application Ser. No. 63/801,244; titled “SURGICAL TRAY TRACKER”; and filed May 7, 2025.

### BACKGROUND

Healthcare facilities utilize large numbers of reusable surgical trays that hold surgical instruments and, in some cases, implants for use during surgical procedures. Surgical trays are commonly processed through a series of steps, including decontamination, washing, sterilization, and storage, before being made available for use in an operating room. Many surgical trays are owned by an implant or instrumentation vendor and provided to a healthcare facility as consignment inventory, such that the vendor retains ownership of the tray until it is used, while the healthcare facility is responsible for its custody and safekeeping while on the facility's premises.

Tracking and documentation within a healthcare facility have historically also relied at least in part on manual logs maintained by facility personnel to record the location, processing history, and custody of surgical trays and other reusable equipment as they move between departments and, in the case of consigned inventory, between the facility and outside vendors. This manual documentation is time-consuming, prone to transcription errors, and difficult to audit or aggregate for quality improvement analysis.

Sterilization equipment such as a sterilizer chamber for sterilizing surgical trays may include sensors that log chamber-level temperature, pressure, or other conditions during a processing cycle, but such monitoring does not verify that each individual tray experienced adequate sterilization conditions. For example, temperature gradients can exist within a sterilizer chamber due to air pockets, dense instrument packs, or suboptimal loading. Thus, a tray may not achieve adequate lethality even though the chamber sensors indicate compliant conditions.

Thus, there exists a need for surgical tray tracking and verification systems and methods that overcome these and other deficiencies of the prior art.

### SUMMARY

Embodiments herein address one or more of the above-mentioned problems and provide a distinct advance in the art of tracking surgical tray locations and procedures throughout workflow cycles and life cycles thereof. In some embodiments, a computer-implemented method for tracking a surgical tray within a healthcare facility includes receiving from a sensor tag coupled to the surgical tray location data and a time series of temperature data, determining a workflow state of the surgical tray based on the location data, and computing a processing-exposure value based on the time series of temperature data. The computer-implemented method further includes generating cryptographic proof indicating that the processing-exposure value satisfies a predetermined threshold, without revealing the time series of temperature data or the processing-exposure value. The cryptographic proof and a chain-of-custody record associated with the surgical tray are recorded on a distributed ledger, which recording may trigger a pre-determined action based at least partially on the generating of or the recording of the cryptographic proof that the processing-exposure value satisfies the predetermined threshold.

In another embodiment, a system for tracking a surgical tray includes one or more processors programmed and/or otherwise set up to receive, from a sensor tag coupled to the surgical tray, location data and a time series of temperature data associated with the surgical tray and compute a processing-exposure value based on the time series of temperature data. The one or more processors may also be programmed or otherwise set up to receive the processing-exposure value and the location data from the gateway device, generate a cryptographic proof that the processing-exposure value satisfies a predetermined threshold, without revealing the time series of temperature data or the processing-exposure value, and determine a workflow state of the surgical tray from among a plurality of predefined workflow states within a healthcare facility, based at least partially on one or both of the location data and the cryptographic proof that the processing-exposure value satisfies the predetermined threshold. The one or more processors are also programmed or otherwise set up to record the cryptographic proof and a time-stamped chain-of-custody record associated with the surgical tray on a distributed ledger.

In yet another embodiment, a non-transitory computer-readable medium stores instructions that, when executed by one or more processors of a computing system, causes the computing system to receive location data associated with a sensor tag coupled to a surgical tray, receive a time series of temperature data from the sensor tag, determine a workflow state of the surgical tray from among a plurality of predefined workflow states within a healthcare facility, based at least partially on the location data, and calculate or compute a processing-exposure value based on the time series of temperature data. The instructions may further cause the computing system to generate a cryptographic proof that the processing-exposure value satisfies a predetermined threshold, without revealing the time series of temperature data or the processing-exposure value, record the cryptographic proof and a time-stamped chain-of-custody record associated with the surgical tray on a distributed ledger, and trigger a pre-determined action in response to the recording of the cryptographic proof indicating that the processing-exposure value satisfies the predetermined threshold. The cryptographic proof is generated via a zero-knowledge proof protocol receiving as private inputs the processing-exposure value and the time series of temperature data, and receiving as public inputs the predetermined threshold and a cryptographic commitment formed by hashing the processing-exposure value in accordance with a cryptographic hash function.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the technology herein will be apparent from the following detailed description of the embodiments and the accompanying drawing figures.

### BRIEF DESCRIPTION OF DRAWINGS

Embodiments of the currently-disclosed technology are described in detail below with reference to the attached drawing figures, wherein:

FIG. 1 is a block diagram of a system for tracking a surgical tray, in accordance with embodiments herein;

FIG. 2 is a block diagram of components of the sensor tag of FIG. 1, in accordance with embodiments herein;

FIG. 3 is a schematic elevation view of an electromechanical lock and storage receptacle for the surgical tray of FIG. 1, in accordance with embodiments herein;

FIG. 4 is a state diagram illustrating a plurality of workflow states through which a surgical tray may transition, in accordance with embodiments herein;

FIG. 5 is a block diagram illustrating notifications communicated among the computing system, a hospital computing device, and a vendor computing device of FIG. 1, in accordance with embodiments herein;

FIG. 6 is a block diagram illustrating a proximity search subsystem for locating a plurality of surgical trays within a healthcare facility, in accordance with embodiments herein;

FIG. 7 is a flow chart of a method of tracking a surgical tray, in accordance with embodiments herein; and

FIG. 8 is a flow chart of a method of tracking a plurality of surgical trays in a case-level sterilization container in accordance with embodiments herein.

The drawing figures do not limit the current disclosure to the specific embodiments disclosed and described herein. The drawings are not necessarily to scale, emphasis instead being placed upon clearly illustrating the principles of the technology.

### DETAILED DESCRIPTION

The following detailed description of the technology references the accompanying drawings that illustrate specific embodiments in which the technology can be practiced. The embodiments are intended to describe aspects of the technology in sufficient detail to enable those skilled in the art to practice the technology. Other embodiments can be utilized and changes can be made without departing from the scope of the technology herein. The following detailed description is, therefore, not to be taken in a limiting sense.

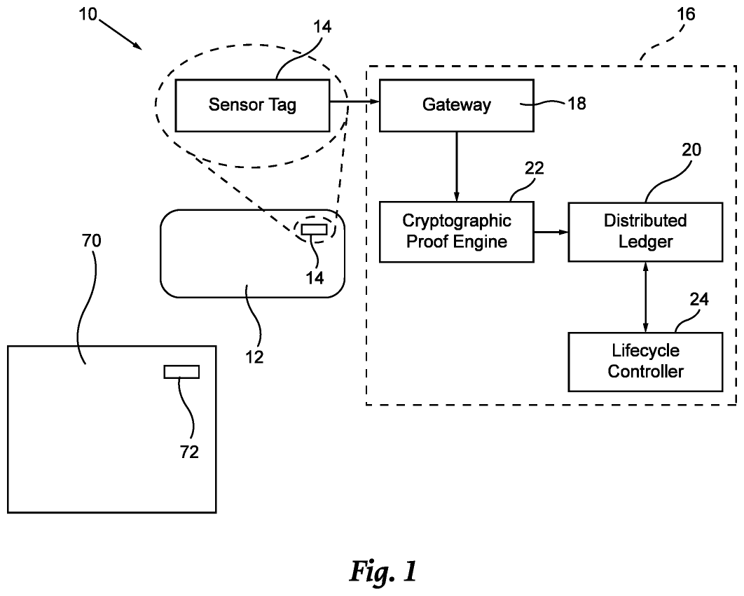

As depicted in FIG. 1, a system 10 is disclosed herein for tracking a surgical tray 12 as it moves through a healthcare facility, for documenting a processing-exposure history and chain-of-custody of the surgical tray 12, and for detecting deviations from an expected workflow. In some embodiments, the system 10 includes a sensor tag 14 attached and/or attachable to the surgical tray 12 and one or more processors 16. Furthermore, in some embodiments, the system 10 includes a gateway device 18, which in some embodiments may be implemented on at least one of the one or more processors 16. The one or more processors 16 can also be configured to implement one or more of the following: a distributed ledger 20, a cryptographic proof engine 22, and/or a lifecycle controller 24. In one or more embodiments, the one or more processors 16 may include a hospital computing device, a vendor computing device, one or more third-party servers, or the like.

The surgical tray 12 may be a rigid component having at least one surface configured for holding and/or organizing medical tools during medical procedures and operations. The surgical tray 12 may be made of surgical-grade stainless steel, medical-grade plastics, or the like. The surgical tray 12 may in some embodiments be perforated or made of wire mesh and/or may comprise built-in slots, compartments, attachment fixtures, and/or smooth edge-free designs for preventing bacteria from hiding in crevices. Alternatively, the systems and methods described herein can be used to track other containers, tools, or items requiring sterilization prior to use without departing from the scope of the technology herein.

Furthermore, in some embodiments, the system 10 may include a case-level sterilization container 70 configured to hold a plurality of surgical trays 12 required for a single surgical procedure, such that all of the surgical trays 12 for the procedure can be sterilized, stored, transported, and presented to an operating room together as a single unit. The case-level sterilization container 70 may be a rigid, filtered sterilization container or another enclosure suitable for maintaining sterility of its contents and may bear a container tag 72. The container tag 72 may be similar or identical to the sensor tag 14 described herein or may alternatively be a simple machine-readable identifier. In some embodiments, the physical case-level sterilization container 70 may hold different ones of the surgical trays 12 from one processing cycle to a next processing cycle, so the association between the case-level sterilization container 70 and its contents is not fixed, but is instead generated and/or updated at various assembly events, as described below.

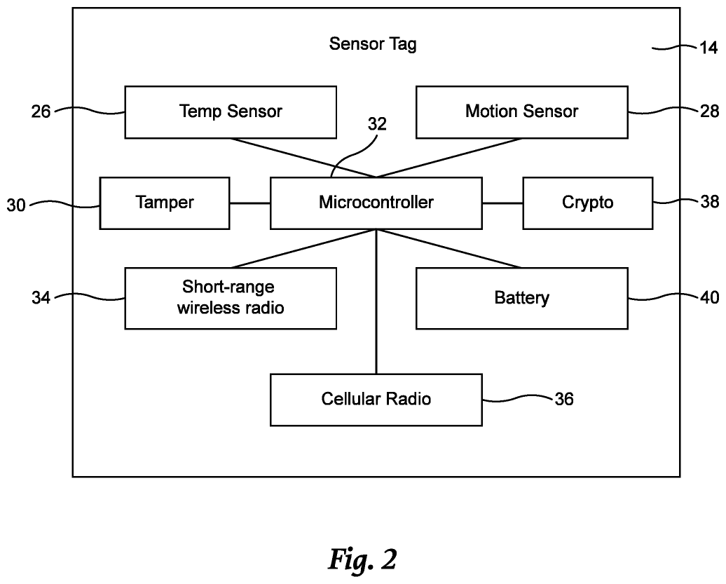

The sensor tag 14 is configured to be coupled to the surgical tray 12 and to generate data usable by the one or more processors 16 to determine a location and/or a processing-exposure value associated with the surgical tray 12. In some embodiments, as depicted in exemplary FIG. 2, the sensor tag 14 includes one or more of a temperature sensor 26, a motion sensor 28, a tamper sensor 30, a microcontroller 32, a short-range wireless radio 34, a cellular radio 36, a cryptographic module 38, and/or a power source 40, any one or more of which may be included in a given embodiment of the sensor tag 14 without requiring inclusion of all such elements. Additional or alternative sensing, processing, or communication components may also be included in the sensor tag 14.

The temperature sensor 26 may be any sensor configured to measure a temperature. For example, the temperature sensor 26 may include any combination of the following: a thermistor, a resistance temperature detector, a thermocouple, or an integrated circuit temperature sensor. The temperature sensor 26 may be communicatively coupled to the microcontroller 32, which may obtain temperature readings therefrom over time to form a time series of temperature data. In some embodiments, the temperature sensor 26 includes more than one temperature sensor. For example, a plurality of temperature sensors may be positioned at different locations relative to the surgical tray 12, such that temperature gradients on the surgical tray 12 may be detected. In such embodiments, for example, F0 calculations as later described herein may use a minimum temperature measured across all sensors, providing a worst-case verification. However, the temperature sensor 26, or a specific type or number thereof, may be varied without departing from the scope of the technology herein.

The motion sensor 28 and the tamper sensor 30, where included, may be configured to detect movement, orientation changes, or unauthorized removal of the sensor tag 14 relative to the surgical tray 12. The motion sensor 28 may include an accelerometer or other inertial sensor, and the tamper sensor 30 may include a mechanical switch, a seal, an electrical continuity sensor, or other component configured to indicate whether the sensor tag 14 has been detached from, or otherwise tampered with relative to, the surgical tray 12. Either or both of the motion sensor 28 and the tamper sensor 30 may be omitted in some embodiments without departing from the scope of the technology herein.

The microcontroller 32 may be located on and/or in the sensor tag and may comprise any electronic controller or processor known in the art, such as any of the processors or processing elements described herein. The microcontroller 32 may be communicably and/or electrically coupled with any of the components of the sensor tag 14. Additionally or alternatively, the microcontroller 32 may be replaced by other computing devices, processors, circuitry, and/or programmable hardware components without departing from the scope of the technology herein.

The short-range wireless radio 34 is configured to wirelessly communicate signal data from the sensor tag 14 to the gateway device 18 using a short-range wireless communication protocol, examples of which include Bluetooth, Bluetooth Low Energy, ultra-wideband, and Wi-Fi. In some embodiments, the sensor tag 14 additionally or alternatively includes the cellular radio 36, configured to wirelessly transmit the signal data from the sensor tag 14 to the one or more processors 16 over a cellular network, independent of the gateway device 18. Use of a combination of the short-range wireless radio 34 and the cellular radio 36 may allow the sensor tag 14 to be located and to communicate the signal data both within and outside of a healthcare facility. However, either the short-range wireless radio 34 or the cellular radio 36 may be omitted in some embodiments without departing from the scope of the technology herein. Furthermore, these components may be replaced with other wireless communication components or wireless transmitters without departing from the scope of the technology herein.

The cryptographic module 38, where included, maybe configured to provide secure key storage and cryptographic functions, such as digitally signing data transmitted from the sensor tag 14, so that data received by the one or more processors 16 may be authenticated as having originated from a particular sensor tag (e.g., the sensor tag 14).

The power source 40 may be a battery or other power storage or generation component configured to provide electrical power to the other components of the sensor tag 14. In some embodiments, the power source 40 and other components of the sensor tag 14 are selected and arranged to withstand repeated exposure to the elevated temperatures and humidity levels encountered during a washing or sterilization process.

In one or more embodiments, the sensor tag 14 may further include a housing configured to be mechanically coupled to the surgical tray 12, using an attachment mechanism. The attachment mechanism may be any of a clip, a bracket, a magnetic coupling, adhesive, or the like. Alternatively, the sensor tag 14 may be integrated into the surgical tray 12 during manufacture thereof. For example, the surgical tray 12 itself may serve as the housing of the sensor tag 14 and/or may have a compartment formed therein into which the sensor tag 14 may be placed. In some embodiments, the housing is configured to remain coupled to the surgical tray 12 on a substantially permanent basis so as to reduce reliance on manual attachment or reattachment of the sensor tag 14 to the surgical tray 12.

The gateway device 18 may comprise one or more processors as described herein and may be configured to wirelessly communicate with the sensor tag 14 (e.g., using the short-range wireless radio 34 thereof) to receive data transmitted by the sensor tag 14 and to relay the received data to the one or more processors 16, such as via a network connection provided by a healthcare facility. In some embodiments, a plurality of gateway devices, similar or identical to the gateway device 18, are positioned at different fixed locations within a healthcare facility, such that a location of the surgical tray 12 may be determined based on which of the plurality of gateway devices receives data from the sensor tag 14, and/or based on a signal strength or time-of-flight measurement associated with such communication. In some embodiments, the gateway device 18 is integrated with, or communicates via, a wireless network access point already present within the healthcare facility. Additionally or alternatively, the gateway device 18 may be implemented on mobile devices carried by sterile processing staff, eliminating the need for fixed gateway device installation. The gateway device 18 may be omitted in embodiments in which the sensor tag 14 communicates directly with the one or more processors 16, such as by way of the cellular radio 36, and/or with other technologies related to geolocating of a sensor as known in the art.

In some embodiments, the gateway device 18 is configured to continuously or periodically scan for sensor tags (e.g., the sensor tag 14) within range. In one or more embodiments, the sensor tag 14 may be configured to transmit a data packet to the gateway device 18 and/or the one or more processors 16 upon completion of a sterilization cycle (e.g., as determined via the microcontroller 32 based on temperature data). The data packet may include one or more of a surgical tray identifier, timestamp, time series of temperature measurements, motion sensor data, tamper detection status, and/or digital signature. Upon receiving this data packet, the gateway device 18 may be configured to verify the digital signature, validate timestamps, check tamper status, and/or perform data integrity checks in response. In some embodiments, the gateway device 18 may then transmit this data to the one or more processors 16 for processing via software and method steps described herein. In some embodiments, the gateway device 18, the one or more processors 16, or various servers associated therewith via the software described herein may integrate with hospital information systems, electronic health records (EHR), surgical scheduling systems, regulatory reporting platforms, and supplier inventory management systems, automatically exchanging data and triggering workflows.

The one or more processors 16 may be communicably coupled with the gateway device 18 (and/or a plurality of gateway devices as described herein), the sensor tag 14, and other various system components described herein. The one or more processors 16 may include one or more processing elements, computers, computing elements, microprocessors, or other circuitry, as well as memory and/or communication components communicatively coupling the one or more processors 16 to each other and/or to other components described herein, including the sensor tag 14, the gateway device 18, and the distributed ledger 20. In some embodiments, the one or more processors 16 may include and/or be communicably coupled with the hospital computing device, the vendor computing device, and/or a plurality of hospital computing devices and/or vendor computing devices. The one or more processors 16 may be individually or cooperatively configured to perform any of the method steps described herein. In some embodiments, one or more of the functions described herein as being performed by the one or more processors 16 may additionally or alternatively be performed by the gateway device 18 or by the sensor tag 14 (e.g., the microcontroller 32).

The one or more processors 16 are configured to receive data from the sensor tag 14, whether directly or via the gateway device 18, and to process such data to determine a location and a workflow state of the surgical tray 12, to compute a processing-exposure value associated with the surgical tray 12, and/or to generate and record a cryptographic proof and chain-of-custody record associated therewith. The one or more processors 16 may comprise one or more processing elements (as described under “Other Considerations” below), one or more servers, cloud-based computing resources, and/or other computing devices, and may include a cryptographic proof engine 22 configured to generate a cryptographic proof as described in more detail below. The one or more processors 16 may include, or be communicatively coupled to, other various processors and memory devices, further details of which are provided under “Other Considerations” below. In some embodiments, as noted above, the one or more processors 16 may include hospital computing devices, vendor computing devices, one or more third-party servers, and/or the like.

The one or more processors 16 may further be configured to communicate with the hospital computing device and the vendor computing device, such as to provide a dashboard or portal by which authorized users associated with a healthcare facility or a vendor entity, respectively, may view location, workflow-state, and chain-of-custody information associated with the surgical tray 12 and/or a plurality of surgical trays. In some exemplary embodiments, the one or more processors 16 may be configured to detect an anomaly associated with the surgical tray and to generate an exception alert responsive thereto. Examples of an anomaly include the surgical tray 12 (and/or the sensor 14 attached thereto) skipping an expected workflow state, the surgical tray 12 remaining in a given workflow state for longer than an expected duration, an indication from the tamper sensor 30 that the sensor tag 14 has been removed or tampered with, a failure of the cryptographic proof engine 22 to generate a valid cryptographic proof (as described below), and removal of the surgical tray 12 from a healthcare facility without an associated authorization record. Upon occurrence of such anomaly, at least some of the one or more processors 16 may transmit an exception alert to one or both of the hospital computing device and the vendor computing device and/or update records stored in memory and accessible by computing devices of the hospital and/or vendor.

In some embodiments, the one or more processors 16 compute the processing-exposure value based on a time series of temperature data received from the sensor tag 14 and a reference temperature associated with a particular processing modality, such as washing, thermal disinfection, or steam sterilization. As one example, the processing-exposure value may be computed as a thermal lethality value analogous to an F0 or A0 value used in connection with steam sterilization or washer-disinfector cycles, respectively, such as by summing, over the time series of temperature data, a term that increases exponentially with the difference between a measured temperature and the reference temperature, as described below. Other computations for the processing-exposure value, including computations associated with other processing modalities such as ethylene oxide, hydrogen peroxide plasma, ozone, or radiation-based processing, may also be used without departing from the scope of the technology herein.

Specifically, in some embodiments, the one or more processors 16 may be configured to calculate or compute the thermal lethality value A0 or F0, using various standards known for steam sterilization cycles and washer-disinfector cycles (e.g., ISO 15883 and/or ISO 17665-1: 2006, respectively). For example, the one or more processors 16 may be configured to compute the processing-exposure value as a thermal lethality value based on a time series of temperature data received from the sensor tag 14 and a predetermined threshold time value associated with sterilization of the surgical tray 12, such as a reference temperature associated with sterilization or disinfection modality for the surgical tray 12. In some alternative embodiments, the thermal lethality values may be calculated or computed by the microcontroller 32 of the sensor tag 14 or at least one of the one or more processors 16.

In an example embodiment for calculating the thermal lethality value A0 for washer-disinfector cycles, the one or more processors 18 may be configured or programmed to solve the following equation:

A0=Σ10\hat{ }((T(t)−80)/10)×Δt

where T(t) is the measured surgical tray temperature at time t in degrees Celsius, ≢t is the time interval between measurements in seconds, the reference temperature is 80 °C, and the z-value is 10 °C. The A0 value represents the equivalent time in seconds at 80 °C. Healthcare standards may require, for example, that A0 ≥600 seconds for adequate thermal disinfection. However, other limts may be used.

Likewise, thermal lethality for steam sterilization cycles may be solved using the following equation

F0=Σ10\hat{ }((T(t)−121.1)/10)×Δt/60

where T(t) is the measured surgical tray temperature in degrees Celsius, Δt is the time interval in seconds, the reference temperature is 121.1 °C, the z-value is 10 °C, and the result is in minutes. The F0 value represents the equivalent sterilization time in minutes at 121.1 °C. In some embodiments, requirements in healthcare may range from F0≥8 minutes to F0≥15 minutes, but may vary and/or extend outside this range depending on the application.

Computing this thermal lethality value from tray-level temperature measurements advantageously provides verification that a specific surgical tray received adequate sterilization rather than relying on chamber-level measurements. While the embodiments herein focus on steam sterilization (F0) and thermal disinfection (A0), the system 10 and methods described herein may also be applied to other sterilization modalities including ethylene oxide (EtO), hydrogen peroxide plasma, ozone, radiation, and others, each with appropriate lethality calculations or computations as known in the art. In such alternative sterilization modalities, other sensors may be incorporated into the sensor tag 14 and used in addition to or in place of the temperature sensors described above. Furthermore, other variables sensed by the sensor tab 14 may be utilized via various equations or processing techniques known in the art to calculate a sterilization time in minutes for those other sensed variables, with such sterilization time used in place of F0 in the calculations below based on amount of gas, radiation, or the like sensed for that amount of time.

The cryptographic proof engine 22 may be implemented via one or more controllers, zero-knowledge proof circuitry, and/or the one or more processors 16 and may be configured to generate a cryptographic proof that the processing-exposure value (e.g., the thermal lethality value) satisfies a predetermined threshold, especially without revealing the time series of temperature data or the processing-exposure value itself. In some embodiments, the cryptographic proof engine 22 implements a zero-knowledge (zk) proof protocol, such as a zero-knowledge succinct non-interactive argument of knowledge (zk-SNARK) or zero-knowledge scalable transparent argument of knowledge (zk-STARK) protocol, taking as private inputs the processing-exposure value (e.g., the thermal lethality value F0), the time series of temperature data T(t), and a random nonce, and taking as public inputs a threshold time value T_min (e.g., F0≥8 minutes) and a cryptographic commitment C formed by hashing the processing-exposure value together with the nonce or using other hashing techniques known in the art. This approach may advantageously allow a healthcare facility to demonstrate that a surgical tray underwent adequate processing conditions without disclosing the underlying temperature data or exact processing-exposure value to a vendor, regulator, or other third party, which data may be considered proprietary or sensitive.

Specifically, in some embodiments, the cryptographic proof engine 22 enforces three constraints: (1) F0 is correctly computed from T(t) according to the standard formula, (2) F0≥T_min using range proof or comparison techniques, and (3) the commitment C is correctly formed as C=Hash(F0||nonce) using a cryptographic hash function such as SHA-256, SHA-3, or Poseidon has, for example. The cryptographic proof engine 22 may be configured to thereby generate a proof π (also referred to herein as the zero-knowledge proof or cryptographic proof) that satisfies all these constraints. The proof can advantageously be verified by anyone without revealing F0, T(t), or the nonce. The proof is also advantageously succinct (e.g., a few hundred bytes) and can be verified efficiently (e.g., in milliseconds). The zero-knowledge proof or cryptographic proof reveals nothing about F0 beyond the fact that F0≥T_min. The actual F0 value, temperature time series, and even the commitment C (when properly hashed) do not reveal sensitive information. This privacy preservation is valuable when hospitals consider sterilization process parameters proprietary, for regulatory compliance requiring verification without unnecessary disclosure, and in multi-party consignment scenarios.

In one or more embodiments, the distributed ledger 20 is configured to store a tamper-evident chain-of-custody record of sterilization verification events associated with the surgical tray 12, as generated by the one or more processors 16. In some embodiments, the chain-of-custody record includes a tray identifier, a timestamp, an indication of a workflow-state transition, and/or the cryptographic proof generated by the cryptographic proof engine 22, without including the time series of temperature data or the processing-exposure value. The distributed ledger 20 may be a public blockchain network, a permissioned or private blockchain or distributed ledger operated by or on behalf of a consortium of healthcare and vendor entities, or another form of tamper-evident distributed data store. In some embodiments, the distributed ledger 20 is a third-party ledger network owned and/or operated by a third party other than an associated hospital or vendor. In one or more embodiments, when a sterilization cycle is verified as having occurred (e.g., the cryptographic proof thereof is generated), a transaction is recorded (e.g., on the distributed ledger 20 or the like). In some embodiments, the transaction items recorded include: surgical tray identifier, timestamp, the zero-knowledge proof π, the cryptographic commitment C, various metadata (e.g., cycle type, facility identifier, operator), and/or a digital signature. The transaction does not include the actual F0 value or temperature data, as the purpose is to track the surgical tray 12 and ensure that it went through a sterilization process, but not to confirm that the surgical tray 12 itself is sterile. Other traditional processes and procedures undertaken by the hospital to ensure safety and complete sterilization would continue to be used, but the system and methods herein would merely provide for tracking of what steps in a workflow have and have not occurred to the surgical tray 12, as well as where in the facility the surgical tray 12 is located.

The hospital computing device and the vendor computing device may each be any computing device, such as a desktop computer, laptop computer, tablet, or smartphone, operated respectively by personnel or employees associated with a healthcare facility and by personnel associated with a vendor entity, such as a sales representative or distributor of surgical trays. In some embodiments, the hospital computing device and/or the vendor computing device may be configured to receive notifications and alerts from, and to query location, workflow-state, and chain-of-custody information from, the one or more processors 16 (e.g., via software executed on the hospital computing device, the vendor computing device, and/or remotely via servers or the like).

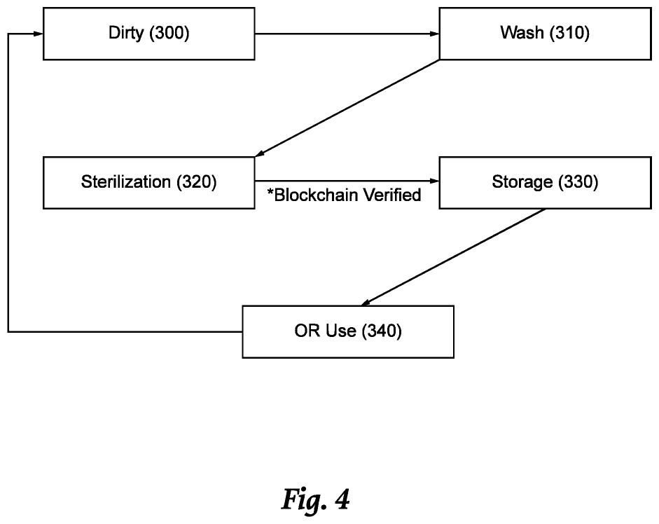

The lifecycle controller 24 may be computer code or instructions implemented on any of the one or more processors 16, such as cloud computing devices and/or a personal computer, smart phone, tablet, or any of the processors described herein. The lifecycle controller 24 may be configured to track a workflow state of the surgical tray 12 from among a plurality of predefined workflow states. For example, as depicted in FIG. 4, workflow states tracked by the lifecycle controller 24 may include a dirty state 300, a wash state 310, a processing state 320, a storage state 330 (e.g., on-site or off-site storage), and an operating-room (OR) use state 340. Additionally or alternatively, in some embodiments the surgical tray 12 may be removed from this workflow and indicated via the lifecycle controller 24 as being in an off-site or checked-out state. However, one or more of these states may be omitted in a workflow and/or other states may be required or utilized for the systems and methods herein without departing from the scope of the technology herein. The lifecycle controller 24 may determine a current workflow state based on or partially based on location data and/or other data received from the sensor tag 14, may enforce an expected sequence of workflow-state transitions, and may gate a given workflow-state transition, or another operational or financial action, on validation of a cryptographic proof recorded on the distributed ledger 20. The lifecycle controller 24 may further track a number of processing cycles undergone by the surgical tray 12, based on the chain-of-custody record, and may generate a replacement notification, for transmission to the vendor computing device and/or the hospital computing device, responsive to the number of processing cycles satisfying a predetermined threshold. Additionally or alternatively, the lifecycle controller 24 may output a ready-for-restock notification after completion of a particular workflow state and/or based on the number of processing cycles undergone.

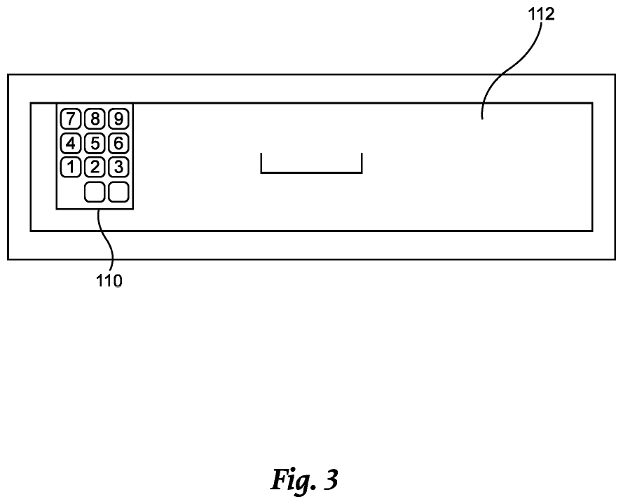

In some embodiments, as depicted in FIG. 3, the system 10 further includes an electromechanical lock 110 coupled to the surgical tray 12 or to a storage receptacle 112 therefor. The electromechanical lock 110 may be configured to be released by receiving a release signal or unlock signal from one or more of the processors 16 responsive to validation of a cryptographic proof recorded on the distributed ledger 20. The electromechanical lock 110 may also be configured, in some embodiments, to provide an indication (e.g., to the one or more processors 16 or a user device) that the surgical tray 12 has left a designated storage location. The electromechanical lock 110 may be omitted in some embodiments without departing from the scope of the technology herein.

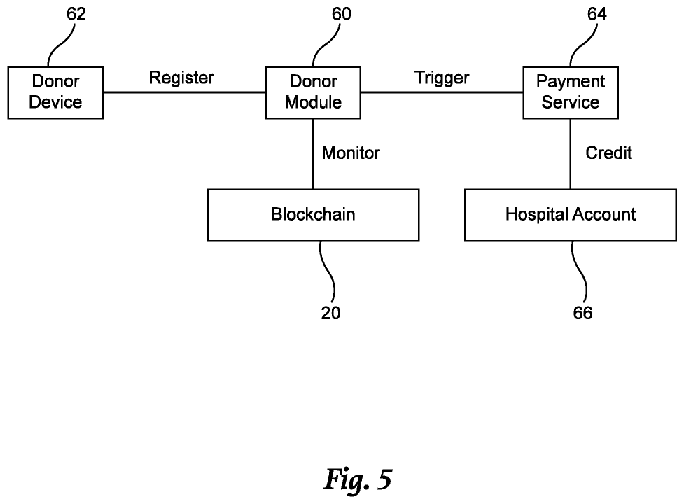

In some embodiments, as depicted in FIG. 5, the lifecycle controller 24 and/or a donor sponsorship module 60 associated therewith may be configured to interact with and/or implement a donor sponsorship program wherein individuals or organizations financially sponsor surgical trays and donation events are triggered automatically when sponsored surgical trays complete verified sterilization cycles. For example, one or more donors may sign up via a donor's electronic device 62 (e.g., a smart phone, tablet, personal computer, or the like), sending account information and associating one or more of the sensor tags 14 and/or their associated surgical trays 12 with the account information. The donor may select a particular amount of money to donate per sterilization cycle, a number of cycles to sponsor, a particular number of surgical trays to sponsor, and/or particular locations or hospitals to which the donor wishes to donate. The donor may also select via the donor's electronic device 62 a payment method (e.g., credit card ACH transfer, cryptocurrency, or the like). The donor sponsorship module 60 may be configured to monitor the distributed ledger 20, and may be configured to trigger payments processed through a payment service 64, donation records may be stored via the distributed ledger 20 for transparency (and/or stored via other databases), and funds may be credited to specific hospital and/or vendor accounts 66 associated with such surgical trays that experienced the triggering event. The funds may even be automatically earmarked for specific departments, such as sterile processing, within the hospital and/or vendor accounts 66. The triggering event may include an indication from the distributed ledger 20 that the sponsored ones of the surgical trays were verified as having completed their sterilization cycles. The transaction may be recorded by documenting via the distributed ledger 20 one or more of: the donation, donor identifier (e.g., could be donor name, organization, and/or anonymous), surgical tray identifier, donation amount, and a corresponding timestamp. The donor sponsorship module 60 may also be configured to output a donor receipt and notification of their contribution to the donor's electronic device 62 (e.g., via email, text message, or mobile app).

The donor sponsorship system described above and depicted in FIG. 5 may enable several beneficial scenarios: grateful patients or families can sponsor surgical trays used in their successful surgeries, providing ongoing support for the hospital's sterilization quality; community members can support healthcare facilities serving underserved populations; corporate sponsors can demonstrate community investment; and memorial or tribute donations can be made in honor of loved ones, with each sterilization cycle serving as a lasting tribute.

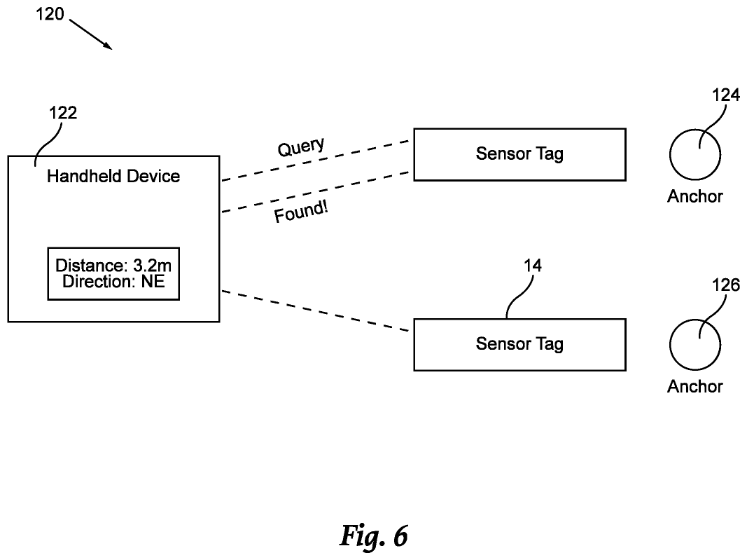

In one or more embodiments, as depicted in FIG. 6, the system 10 further includes a proximity search subsystem 120 configured to assist a user in locating a specific surgical tray within a healthcare facility. In some embodiments, the proximity search subsystem 120 includes a handheld device 122 configured to wirelessly query the sensor tag 14 and to determine a distance and/or direction to the surgical tray 12 based on a signal strength or time-of-flight measurement associated with a response received from the sensor tag 14. In some embodiments, the proximity search subsystem 120 further includes one or more anchor points 124, 126 installed at known locations within the healthcare facility to enable triangulation-based positioning of the surgical tray 12. In some embodiments, the anchor points 124,126 may comprise a plurality of gateway devices, such as the gateway device 18 described above.

In use, a method of tracking a surgical tray with the system 10 or other such systems may include the steps of receiving from the sensor tag 14 location data and/or a time series of temperature data associated with the surgical tray 12; determining a workflow state of the surgical tray 12 based on the location data and/or other such data received from the sensor tag 14; computing a processing-exposure value based on the time series of temperature data; generating a cryptographic proof that the processing-exposure value satisfies a predetermined threshold, without revealing the time series of temperature data or the processing-exposure value; and recording the cryptographic proof and/or a chain-of-custody record associated with the surgical tray 12 on the distributed ledger 20. In some embodiments, the method further includes generating notifications and/or triggering particular actions responsive to verification of a particular workflow state (e.g., cryptographic proof that the processing-exposure value satisfies the predetermined threshold, detecting that the workflow state deviated from an expected sequence of workflow states, and/or detection of another anomaly).

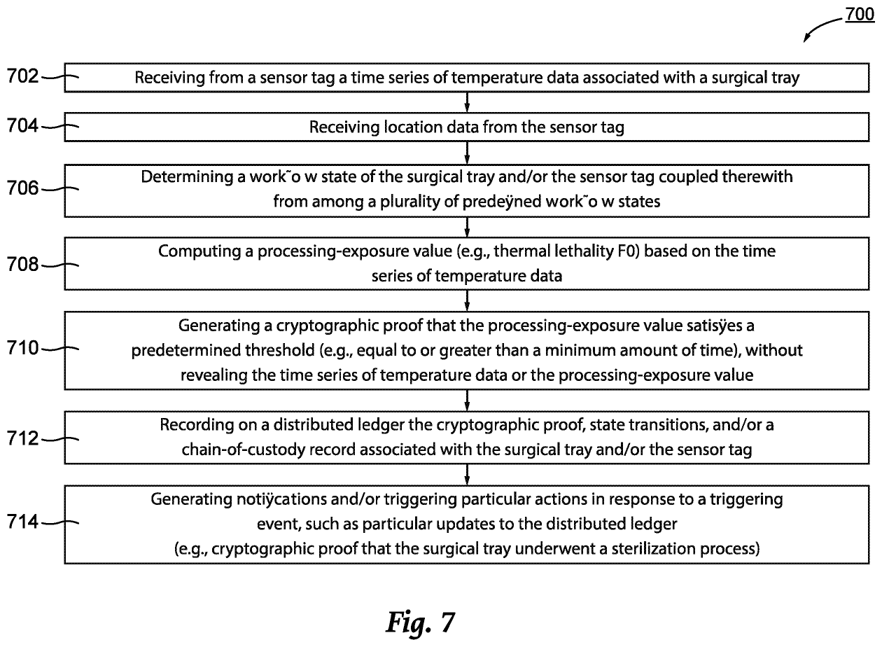

A flow chart of FIG. 7 depicts an exemplary method 700 of tracking a surgical tray in more detail. In some embodiments, various steps may be omitted, or steps may occur out of the order depicted in FIG. 7 without departing from the scope of the technology as described herein. For example, two blocks shown in succession in FIG. 7 may in fact be executed substantially concurrently, or blocks may sometimes be executed in the reverse order depending upon the functionality involved.

In some embodiments, the method 700 includes a step of receiving from a sensor tag (e.g., the sensor tag 14 coupled to the surgical tray 12) a time series of temperature data associated with the surgical tray 12, as depicted in block 702. The method 700 may further include a step of receiving location data from the sensor tag, as depicted in block 704. For example, the location data may be received directly or via the gateway device 18 as described above. That is, the location data may be received from the signal data output by one or more gateway devices disposed at fixed locations within the healthcare facility. The signal data may be based on short-range wireless communication (e.g., Bluetooth or the like) between the sensor tag 14 and the one or more gateway devices 18.

In some embodiments, the method 700 further includes a step of determining a workflow state of the surgical tray 12 and/or the sensor tag 14 coupled therewith from among a plurality of predefined workflow states, as depicted in block 706. For example, the workflow state may be determined based on the location data and the sequency of particular location data received.

In some embodiments, the method 700 further includes a step of computing a processing-exposure value based on the time series of temperature data, as depicted in block 708 and generating a cryptographic proof that the processing-exposure value satisfies a predetermined threshold, without revealing the time series of temperature data or the processing-exposure value, as depicted in block 710. In some embodiments, the one or more processors 16 perform the computing in block 708, such as a remote server or computing devices on which the software or computer instructions described herein are executed. The same or others of the one or more processors 16 may generate the cryptographic proof using the processing-exposure value. For example, the remote server or other ones of the processors 16 may receive, compute, and/or calculate the processing-exposure value and generate a cryptographic proof that the processing-exposure value satisfies a predetermined threshold, without revealing the time series of temperature data or the processing-exposure value to any user or to any of the memory storage related therewith. As described in detail above, the cryptographic proof may be generated via a zero-knowledge proof protocol receiving as private inputs the processing-exposure value and the time series of temperature data (as well as a nonce in some embodiments), and receiving as public inputs the predetermined threshold (e.g., the threshold time value) and a cryptographic commitment formed by hashing the processing-exposure value together with a nonce (and/or using other hashing techniques known in the art). However, other cryptographic proof protocols maybe used without departing from the scope of the technology herein.

In some embodiments, the method 700 further includes a step of recording the cryptographic proof and/or updating a chain-of-custody record associated with the surgical tray 12 (and/or the sensor tag 14) on a distributed ledger, as depicted in block 712, but not recording the time series of the temperature data and/or not recording the processing-exposure value (e.g., the thermal lethality value). For example, upon generation of the cryptographic proof that the processing-exposure value satisfies the predetermined threshold, the cryptographic commitment C derived from the time series of temperature data may be stored rather than the temperature data or the processing-exposure value itself. Specifically, as described above, the cryptographic commitment C may be obtained by hashing the processing-exposure value (which is derived from the time series of temperature data) together with the nonce (which may be a private input). In some embodiments, the one or more processors 16 and/or the lifecycle controller 24 may ensure that all state transitions are logged on the distributed ledger 20 as well, in addition to the validation via the cryptographic proof that the surgical tray 12 has undergone the sterilization process. Furthermore, in some embodiments, the lifecycle controller 24 may query the distributed ledger 20 to verify sterilization via the zero-knowledge or cryptographic proofs before permitting gated transitions to a next state in the workflow for the surgical tray 12.

In some embodiments, the method 700 further includes an additional step of triggering a pre-determined action in response to a triggering event, as depicted in block 714. For example, the triggering event may be receiving or determining verification of a particular workflow state, validating (e.g., via the cryptographic proof) that the processing-exposure value satisfies the predetermined threshold (e.g., updating of the distributed ledger as in block 712 may be the triggering event in some embodiments), detecting that the workflow state deviated from an expected sequence of workflow states, and/or detection of another anomaly. For example, the surgical tray 12 and/or the sensor tag 14 being located in an unauthorized or unexpected location may be a triggering event and/or prior to the cryptographic proof that the surgical tray 12 underwent a sterilization process. Other anomalies may include the surgical tray skipping workflow states, receiving indication of tampering from the sensor tag 14, or an indication of verification failure for the sensor tag 14. In some embodiments, the pre-determined action may include one or more of an operational action or reaction (e.g., unlocking the electromechanical lock 110), a lifecycle transition and/or workflow transition of the surgical tray 12, and/or a financial transaction associated with the surgical tray 12.

The pre-determined action may be generating notifications and sending them to computing devices of hospital personnel, vendors, or the like. For example, some notifications may require the hospital personnel to track down a missing one of the surgical trays and/or notifies the hospital or vendor when the surgical tray 12 has undergone or is approaching a maximum number of uses/cycles for that particular surgical tray and thus should be removed from use. Specifically, the triggering step may include, based on the chain-of-custody record associated with the surgical tray, generating a replacement notification responsive to a number of cycles undergone by the surgical tray 12 satisfying a predetermined threshold (e.g., the maximum number of uses/cycles). In some embodiments, the particular actions triggered in the step of block 714 may include the donations described above in reference to FIG. 5 or other automated financial transactions based on the workflow state, the cryptographic proof, or other data recorded and tracked herein.

Additionally or alternatively, the actions triggered in the step of block 714 may be related to live cycle management and gating (e.g., verifying of particular conditions before permitting a next lifecycle state). For example, the electromechanical lock 110 may be coupled to the surgical tray 12 and/or a storage cabinet storing the surgical tray 12, and the electromechanical lock 110 is released only upon verification of the cryptographic proof. In other example embodiments, the action triggered in the step of block 714 may be related to gating the transition from one state to another, such as from “in storage” and “in use” states based on verification that the surgical tray is in a sterile state. The sterile state may for example require an indication that the cryptographic proof was recorded (e.g., that the sterilization step has occurred for the surgical tray) and that the surgical tray is still within an expiration window of time (e.g., 7-30 days in some embodiments). In some example embodiments, once the surgical tray has been requested for a scheduled surgical case, which may be considered a triggering event, the electromechanical lock 110 may automatically unlock if the surgical tray 12 is in the sterile state.

Furthermore, in some embodiments, the action triggered may be a payment from a hospital to a vendor and/or from a donor to the hospital or the vendor. For example, in one embodiment, each surgical tray has an associated use counter (implemented via components of the sensor tag 14 and/or via the one or more processors 16) that increments only after validated cryptographic proof that the sterilization process occurred. The use counter may be configured to drive a pay-per-clean billing model where hospitals pay suppliers or vendors a fee for each verified sterilization cycle rather than purchasing trays outright. Specifically, upon successful verification (e.g., validated blockchain verification via the distributed ledger 20), the use counter increments, a billing event is triggered, and payment processing is initiated between the hospital entity and supplier or vendor entity. This advantageously provides automated, transparent billing based on cryptographically verified usage.

Conversely, in an example consignment inventory management model, ownership of surgical trays remains assigned to a supplier entity until actual use. The cryptographic proof/blockchain verification provides the trigger for ownership transfer or usage fee calculation. Before verification, surgical trays remain on the vendor's balance sheet. After verification and use, transfer of ownership or billing to the hospital or surgical center may occur. Furthermore, in some embodiments, a digital tray token is recorded on the distributed ledger 20/blockchain and associated with each surgical tray's identifier. The digital token represents ownership rights, consignment status, usage rights, or other property interests in the surgical tray 12. The token may implement standards such as ERC-721 (NFT) or ERC-1155 on Ethereum-based systems, or equivalent token standards on other blockchain platforms. Additionally or alternatively, in some embodiments, validation of the zero-knowledge proof triggers execution of a smart contract associated with the digital token. The smart contract may distribute value between the vendor entity and the healthcare entity according to predetermined terms, update ownership records, process royalty or license payments, or execute other programmed financial logic. In some embodiments using smart contract platforms, a smart contract implements verification and gating logic, stores verification events indexed by tray identifier, manages lifecycle state transitions, and triggers events when verification is recorded. The smart contract may in some example embodiments include logic to verify the zero-knowledge proof either on-chain (e.g., public ledger) or may allow off-chain (e.g., private ledger) verification by any party.

Additionally or alternatively, in some embodiments herein, a method of interacting with the system 10 by hospital personnel (e.g., via the hospital computing device) may include the steps of receiving a workflow-state notification indicating deviation from an expected sequence and/or some anomaly, and querying the proximity search subsystem 120 to locate the surgical tray 12. Similarly, in some embodiments, a method of interacting with the system 10 by vendor personnel (e.g., via the vendor computing device) may include the steps of receiving a restock or replacement notification from the one or more processors 16, and accessing a chain-of-custody record associated with the surgical tray via the one or more processors 16.

The systems and methods described herein may provide several advantages relative to prior tracking and documentation approaches. Automated, gateway-and/or cellular-based location tracking may allow a surgical tray to be located within a healthcare facility with greater granularity and less manual effort than approaches requiring a user to manually scan or retrieve a tracking device. Automated computation of a processing-exposure value and generation of a corresponding cryptographic proof may allow processing history to be documented without manual recordkeeping, while a distributed ledger may provide a tamper-evident chain-of-custody record accessible to multiple stakeholders, including a healthcare facility and a vendor entity. Use of a cryptographic proof that a processing-exposure value satisfies a threshold, without revealing the underlying temperature data or the processing-exposure value itself, may allow such verification to occur without requiring disclosure of process parameters that a healthcare facility may consider proprietary. Automated exception alerts may further reduce the risk that a deviation from an expected workflow (e.g., a skipped processing step or workflow state or an unauthorized removal of a surgical tray from a healthcare facility) goes unnoticed.

It is noted that the processing-exposure value and cryptographic proof described herein document that a surgical tray was exposed to defined processing conditions and are not intended to constitute a determination or guarantee of sterility of the surgical tray. The systems and methods disclosed herein are designed for use in tracking, not proof of sterility. Thus, it is anticipated that healthcare facilities will continue to apply their own protocols, including biological indicator testing, in determining whether a surgical tray is suitable for clinical use.

Although useful for tracking surgical trays as described above, the system 10 and methods herein may also be used for tracking other reusable surgical instrumentation, implant trays, loaner or consignment inventory, and other reusable medical equipment that is transported among processing, storage, and use locations within a healthcare facility, or between a healthcare facility and a vendor or supplier entity. Furthermore, as described above, the case-level sterilization container 70 may be tracked via the container tag 72 in a same or identical manner as the steps described above. For example, the one or more processors 16 may be configured to receive and/or derive a case manifest identifying a plurality of the surgical trays 12 required for a scheduled surgical procedure. The case manifest can be stored and/or retrieved by the one or more processors 16, or may be derived from a surgical scheduling system, an electronic health record, a surgeon preference card, and/or user input via the hospital computing device. The case manifest may serve as a bill of materials for the case-level sterilization container 70. In use, the relevant ones of the surgical trays 12 are loaded into the case-level sterilization container 70, and the detected location of the sensor tags 14 associated with those surgical trays 12 may be within a particular threshold distance range from the container tag 72 of the case-level sterilization container 70. Based on this location data and/or user input, the one or more processors 16 may compare detected identifiers of the sensor tags 14 coupled to the loaded surgical trays 12 against the case manifest. For example, when the sensor tags 14 of each of the surgical trays 12 identified on the case manifest are determined to be within the threshold distance of the container tag 72, this may indicate to the one or more processors 16 that the sterilization container 70 is in a loading-complete state or a case-ready state, as described below. Upon verification that each of the surgical trays 12 identified in the case manifest is present within the case-level sterilization container 70, the one or more processors 16 may record an assembly record on the distributed ledger 20, the assembly record associating an identifier of the container tag 72 with the identifiers of the detected sensor tags 14, together with a timestamp and, in some embodiments, one or more identifiers of personnel performing the assembly or loading of the surgical trays 12. In this manner, the chain-of-custody records described herein extend to the case-level sterilization container 70, and any physical container may be associated with any set of surgical trays 12 while the distributed ledger 20 maintains an independent, auditable association history for each container and tray.

In some embodiments, other verifications of completeness may be performed at multiple nested levels. For example, each instrument, implant, or other item held by a given surgical tray 12 may bear its own identifier (for example, an additional sensor tag 14, a passive machine-readable tag, or other identifier), and each surgical tray 12 may be associated in memory accessible to the one or more processors 16 with a tray manifest defining the required contents of that surgical tray 12, analogous to a count sheet and used as described above in reference to the case manifest. At assembly or loading (and/or prior to the scheduled surgical procedure), the one or more processors 16 may be configured to verify item-to-tray completeness against the tray manifest, tray-to-container completeness against the case manifest, and thereby container-to-surgical case completeness for the scheduled surgical procedure.

In some embodiments, responsive to determining that one of the surgical trays 12 identified in the case manifest is not detected within the case-level sterilization container 70, or that one of the surgical trays 12 is incompletely stocked relative to its tray manifest (e.g., due to a missing, damaged, expired, or count-discrepant item), the one or more processors 16 may be configured to generate a deficiency notification. The deficiency notification is transmitted to a computing device of a responsible party identified in a responsible-party record (e.g., as stored in memory) associated with the deficient surgical tray 12. In some embodiments, the responsible-party record may identify the responsible party based on an ownership attribute of the surgical tray 12. For example, the deficiency notification may be transmitted to a computing device of sterile processing personnel of the healthcare facility for a facility-owned surgical tray, or to the vendor computing device for a loaner, consignment, or implant surgical tray owned by a vendor entity, consistent with the consignment inventory arrangements described above. The deficiency notification may identify the deficient surgical tray, the specific missing or deficient item or items, a last-known location of the deficient surgical tray or item as determined from the location data and/or the proximity search subsystem 120, and/or the surgical procedure and time by which the deficiency must be resolved. Transmitting the deficiency notification directly to the responsible party in this manner advantageously allows deficiencies discovered at assembly to be resolved before sterilization of the case-level sterilization container 70, rather than being discovered in the operating room.

In some embodiments, the one or more processors 16 are further configured to track resolution of the deficiency notification to completion. Upon restocking or replacement of the deficient item, the one or more processors 16 may be configured to verify that the tray manifest and the case manifest are satisfied before updating the assembly record on the distributed ledger 20. In some embodiments, the one or more processors 16 may be configured to escalate the deficiency notification to a supervisor computing device or an alternate responsible party responsive to the deficiency remaining unresolved within a configurable time period before the scheduled surgical procedure.

In some embodiments, the lifecycle controller 24 maintains a case-ready state for the case-level sterilization container 70 and gates entry into the case-ready state on satisfaction of a plurality of conditions, including one or more of: (a) verification that the case manifest and each applicable tray manifest are satisfied; (b) recording on the distributed ledger 20 of a cryptographic proof, generated as described above, that the processing-exposure value associated with each surgical tray 12 within the case-level sterilization container 70 satisfies the predetermined threshold; and (c) absence of any post-sterilization anomaly associated with the case-level sterilization container 70, such as a tamper indication from the container tag 72 or an expiry event. Accordingly, in some embodiments, the case-ready state may be presented to operating room personnel via the hospital computing device as a single readiness indicator for the surgical procedure, in place of individual statuses for each of the plurality of surgical trays 12.

In some embodiments, where the case-level sterilization container 70 attenuates short-range wireless communication between the gateway device 18 and the sensor tags 14 disposed therein, the one or more processors 16 may be configured to resolve the contents of the case-level sterilization container 70 by detecting the container tag 72 and retrieving the associated assembly record from the distributed ledger 20, such that content verification and location tracking remain available without opening the case-level sterilization container 70. Similarly, when a surgical tray 12 identified in the case manifest is determined to be sealed within a different case-level sterilization container, the one or more processors 16 may be configured to resolve the location of that surgical tray 12 (via the prior assembly record associating it with the other container) and report the location of the other container using one or more of the notification or reporting methods or techniques described herein.

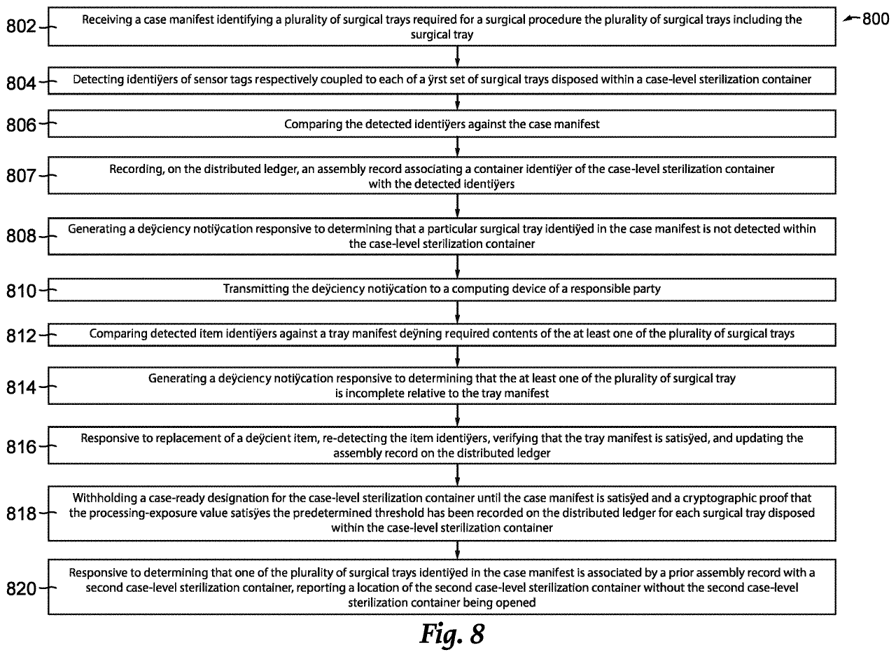

In one example computer-implemented method 800, as depicted in FIG. 8, the method 800 includes the steps of receiving a case manifest identifying a plurality of surgical trays required for a surgical procedure the plurality of surgical trays including the surgical tray 12, as depicted in block 802, detecting identifiers of sensor tags respectively coupled to each of a first set of surgical trays disposed within a case-level sterilization container, as depicted in block 804, comparing the detected identifiers against the case manifest, as depicted in block 806, and recording, on the distributed ledger, an assembly record associating a container identifier of the case-level sterilization container with the detected identifiers, as depicted in block 807.

In some embodiments, the method 800 may include the steps of generating a deficiency notification responsive to determining that a particular surgical tray identified in the case manifest is not detected within the case-level sterilization container, as depicted in block 808, and transmitting the deficiency notification to a computing device of a responsible party, as depicted in block 810. For example, the one or more processors 16 may be configured to select the responsible party from among personnel of a healthcare facility and/or a vendor entity based on an ownership attribute associated with the particular surgical tray that is not detected. In some embodiments, the deficiency notification identifies the surgical tray that is not detected, a last-known location thereof determined based on location data received from the sensor tag coupled thereto, and/or a time by which the deficiency must be resolved based on a scheduled time of the surgical procedure. Responsive to the deficiency remaining unresolved within a configurable time period before the surgical procedure, the method 800 step 810 above may further include escalating the deficiency notification to a computing device of a supervisor or an alternate responsible party.

Likewise, in embodiments where each of a plurality of items held by at least one of the plurality of surgical trays bears an item identifier, the method 800 further includes the steps of comparing detected item identifiers against a tray manifest defining required contents of the at least one of the plurality of surgical trays, as depicted in block 812, and generating a deficiency notification responsive to determining that the at least one of the plurality of surgical tray is incomplete relative to the tray manifest, as depicted in block 814.

Furthermore, the method 800 may include a step of, responsive to replacement of a deficient item, re-detecting the item identifiers, as depicted in block 816, verifying that the tray manifest is satisfied, as depicted in block 818, and updating the assembly record on the distributed ledger, as depicted in block 820.

Furthermore, in some embodiments, the method 800 may include the step of withholding a case-ready designation for the case-level sterilization container until the case manifest is satisfied and a cryptographic proof that the processing-exposure value satisfies the predetermined threshold has been recorded on the distributed ledger for each surgical tray disposed within the case-level sterilization container, as depicted in block 822. In one or more embodiments, the method 800 may include, responsive to determining that one of the plurality of surgical trays identified in the case manifest is associated by a prior assembly record with a second case-level sterilization container, reporting a location of the second case-level sterilization container without the second case-level sterilization container being opened, as depicted in block 824.

One or more steps of method 800 may be combined with the method 700 without departing from the scope of the technology herein. Furthermore, the method 800 may omit one or more steps and/or perform various ones of the steps in a different order without departing from the scope of the technology herein.

### Other Considerations

Throughout this specification, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the current technology can include a variety of combinations and/or integrations of the embodiments described herein.

Although the present application sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of routines, subroutines, applications, or instructions. These may constitute either software (e.g., code embodied on a machine-readable medium, a non-transitory computer-readable medium, or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as computer hardware that operates to perform certain operations as described herein.

In various embodiments, computer hardware, such as a processing element of the controller, may be implemented as special purpose or as general purpose. For example, the processing element may comprise dedicated circuitry or logic that is permanently configured, such as an application-specific integrated circuit (ASIC), or indefinitely configured, such as an FPGA, to perform certain operations. The processing element may also comprise programmable logic or circuitry (e.g., as encompassed within a general-purpose processor or other programmable processor) that is temporarily configured by software to perform certain operations. It will be appreciated that the decision to implement the processing element as special purpose, in dedicated and permanently configured circuitry, or as general purpose (e.g., configured by software) may be driven by cost and time considerations.

Accordingly, the term “processing element” or equivalents should be understood to encompass a tangible entity, be that an entity that is physically constructed, permanently configured (e.g., hardwired), or temporarily configured (e.g., programmed) to operate in a certain manner or to perform certain operations described herein. Considering embodiments in which the processing element is temporarily configured (e.g., programmed), each of the processing elements need not be configured or instantiated at any one instance in time. For example, where the processing element comprises a general-purpose processor configured using software, the general-purpose processor may be configured as respective different processing elements at different times. Software may accordingly configure the processing element to constitute a particular hardware configuration at one instance of time and to constitute a different hardware configuration at a different instance of time.

Computer hardware components, such as communication elements, memory elements, processing elements, and the like, may provide information to, and receive information from, other computer hardware components. Accordingly, the described computer hardware components may be regarded as being communicatively coupled. Where multiple of such computer hardware components exist contemporaneously, communications may be achieved through signal transmission (e.g., over appropriate circuits and buses) that connect the computer hardware components. In embodiments in which multiple computer hardware components are configured or instantiated at different times, communications between such computer hardware components may be achieved, for example, through the storage and retrieval of information in memory structures to which the multiple computer hardware components have access. For example, one computer hardware component may perform an operation and store the output of that operation in a memory device to which it is communicatively coupled. A further computer hardware component may then, at a later time, access the memory device to retrieve and process the stored output. Computer hardware components may also initiate communications with input or output devices, and may operate on a resource (e.g., a collection of information).

The various operations of example methods described herein may be performed, at least partially, by one or more processing elements that are temporarily configured (e.g., by software) or permanently configured to perform the relevant operations. Whether temporarily or permanently configured, such processing elements may constitute processing element-implemented modules that operate to perform one or more operations or functions. The modules referred to herein may, in some example embodiments, comprise processing element-implemented modules.

Similarly, the methods or routines described herein may be at least partially processing element-implemented. For example, at least some of the operations of a method may be performed by one or more processing elements or processing element-implemented hardware modules. The performance of certain of the operations may be distributed among the one or more processing elements, not only residing within a single machine, but deployed across a number of machines. In some example embodiments, the processing elements may be located in a single location (e.g., within a home environment, an office environment or as a server farm), while in other embodiments the processing elements may be distributed across a number of locations.

Unless specifically stated otherwise, discussions herein using words such as “processing,” “computing,” “calculating,” “determining,” “presenting,” “displaying,” or the like may refer to actions or processes of a machine (e.g., a computer with a processing element and other computer hardware components) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The patent claims at the end of this patent application are not intended to be construed under 35 U.S.C. § 112(f) unless traditional means-plus-function language is expressly recited, such as “means for” or “step for” language being explicitly recited in the claim(s).

Although the technology has been described with reference to the embodiments illustrated in the attached drawing figures, it is noted that equivalents may be employed and substitutions made herein without departing from the scope of the technology as recited in the claims.

Having thus described various embodiments of the technology, what is claimed as new and desired to be protected by Letters Patent includes the following:

1. A computer-implemented method for tracking a surgical tray, comprising:
receiving from a sensor tag coupled to the surgical tray a time series of temperature data associated with the surgical tray;
computing a processing-exposure value based on the time series of temperature data;
generating a cryptographic proof that the processing-exposure value satisfies a predetermined threshold associated with sterilization of the surgical tray, without revealing the time series of temperature data or the processing-exposure value; and
recording the cryptographic proof associated with the surgical tray on a distributed ledger; and
triggering a pre-determined action based at least partially on the generating of or the recording of the cryptographic proof that the processing-exposure value satisfies the predetermined threshold.

2. The computer-implemented method of claim 1, further comprising receiving location data from the sensor tag and determining a workflow state of the surgical tray from among a plurality of predefined workflow states within a healthcare facility, based at least partially on the location data, wherein the triggering is further based on the workflow state of the surgical tray.

3. The computer-implemented method of claim 2, wherein the receiving of the location data comprises receiving signal data from one or more gateway devices disposed at fixed locations within the healthcare facility, wherein the signal data is based on short-range wireless communication between the sensor tag and the one or more gateway devices.

4. The computer-implemented method of claim 2, wherein the pre-determined action is a lifecycle transition, operational action, or financial action associated with the surgical tray.

5. The computer-implemented method of claim 2, further comprising generating a notification responsive to determining that the workflow state deviates from an expected sequence of the plurality of predefined workflow states.

6. The computer-implemented method of claim 1, wherein the generating of the cryptographic proof is performed using a zero-knowledge proof protocol configured to receive as private inputs the processing-exposure value and the time series of temperature data, and to receive as public inputs the predetermined threshold and a cryptographic commitment formed by hashing the processing-exposure value in accordance with a hash function.

7. The computer-implemented method of claim 1, wherein computing the processing-exposure value comprises computing a thermal lethality value based on the time series of temperature data and a reference temperature.

8. The computer-implemented method of claim 1, wherein the triggering comprises processing a payment associated with the sterilization of the surgical tray in response to the generating of or the recording of the cryptographic proof that the processing-exposure value satisfies the predetermined threshold.

9. The computer-implemented method of claim 1, wherein the recording of the cryptographic proof includes updating a chain-of-custody record associated with the surgical tray, wherein the triggering of the pre-determined action includes, based on the chain-of-custody record associated with the surgical tray, generating a replacement notification responsive to a number of processing cycles satisfying a predetermined threshold.

10. The computer-implemented method of claim 1, wherein the triggering of the pre-determined action comprises instructing release of an electromechanical lock associated with at least one of the surgical tray and a storage receptacle therefor, responsive to the recording of the cryptographic proof.

11. The computer-implemented method of claim 1, further comprising:
receiving a case manifest identifying a plurality of surgical trays required for a surgical procedure, the plurality of surgical trays including the surgical tray;
detecting identifiers of sensor tags respectively coupled to a first set of surgical trays disposed within a case-level sterilization container;
comparing the detected identifiers against the case manifest; and
recording, on the distributed ledger, an assembly record associating an container identifier of the case-level sterilization container with the detected identifiers of the first set of surgical trays.

12. The computer-implemented method of claim 11, further comprising generating a deficiency notification responsive to determining that at least one of the plurality of surgical trays identified in the case manifest is not detected within the case-level sterilization container.

13. A system for tracking a surgical tray, the system comprising:
one or more processors, communicatively coupled to a sensor tag coupled to the surgical tray, the one or more processors configured to:
receive from the sensor tag, location data and a time series of temperature data associated with the surgical tray;
compute a processing exposure value based on the time series of temperature data;
generate a cryptographic proof that the processing-exposure value satisfies a predetermined threshold, without revealing the time series of temperature data or the processing-exposure value;
determine a workflow state of the surgical tray from among a plurality of predefined workflow states within a healthcare facility, based at least partially on one or both of the location data and the cryptographic proof that the processing-exposure value satisfies the predetermined threshold; and
record the cryptographic proof and a chain-of-custody record associated with the surgical tray on a distributed ledger.

14. The system of claim 13, wherein the one or more processors are further configured to compute the processing-exposure value as a thermal lethality value based on the time series of temperature data and a reference temperature associated with a sterilization or disinfection modality.

15. The system of claim 13, further comprising the sensor tag, wherein the sensor tag further comprises one or more of a motion sensor, a tamper sensor, a temperature sensor, and a cryptographic module.

16. The system of claim 13, wherein the one or more processors are further configured to detect an anomaly comprising at least one of a skipped workflow state, a tamper indication, and a failure to validate the cryptographic proof, and to generate an exception alert responsive to the anomaly.

17. The system of claim 13, wherein the one or more processors further comprise a lifecycle controller configured to send a notification to a computing device of a vendor or personnel of the healthcare facility in response to a change in the workflow state of the surgical tray.

18. The system of claim 13, wherein the chain-of-custody record comprises a tray identifier, a timestamp, and the cryptographic proof, without the time series of temperature data or the processing-exposure value.

19. The system of claim 13, wherein the one or more processors are further configured to transmit a release signal, in response to validation of the cryptographic proof, to an electromechanical lock coupled to at least one of the surgical tray and a storage receptacle therefor.

20. A non-transitory computer-readable medium storing instructions that, when executed by one or more processors of a computing system, cause the computing system to:
receive location data associated with a sensor tag coupled to a surgical tray;
receive a time series of temperature data from the sensor tag;
determine a workflow state of the surgical tray from among a plurality of predefined workflow states within a healthcare facility, based at least partially on the location data;
calculate a processing-exposure value based on the time series of temperature data;
generate a cryptographic proof that the processing-exposure value satisfies a predetermined threshold, without revealing the time series of temperature data or the processing-exposure value, wherein the cryptographic proof is generated via a zero-knowledge proof protocol receiving as private inputs the processing-exposure value and the time series of temperature data, and receiving as public inputs the predetermined threshold and a cryptographic commitment formed by hashing the processing-exposure value in accordance with a cryptographic hash function;
record the cryptographic proof and a time-stamped chain-of-custody record associated with the surgical tray on a distributed ledger; and
trigger a pre-determined action in response to the recording of the cryptographic proof indicating that the processing-exposure value satisfies the predetermined threshold.
